(19) United States
(12) Patent Application Publication
(10) Pub. No.: US 2024/0261196 A1
(43) Pub. Date: Aug. 8, 2024

(54) COMPOSITION FOR MAKE-UP USE, COMPRISING FLAKE-TYPE ALUMINUM PIGMENTS AND FLAKE-TYPE BLACK PIGMENTS

(71) Applicant: L'OREAL, PARIS (FR)

(72) Inventors: Fumiyoshi IKKAI, Kawasaki-shi (JP); Rena HAYASHI, Kawasaki-shi (JP); Hoshiko HORI, Kawasaki-shi (JP)

(21) Appl. No.: 18/567,495

(22) PCT Filed: Jun. 3, 2022
(86) PCT No.: PCT/JP2022/023391

§ 371 Date: Dec. 6, 2023

(30) Foreign Application Priority Data
Jun. 8, 2021 (JP) 2021-095881
Jul. 5, 2021 (FR) 2107245

(51) Int. Cl. A61K 8/02; A61K 8/19; A61K 8/25; A61K 8/26; A61Q 1/06; A61Q 1/08; A61Q 1/10; A61Q 1/12

(52) U.S. Cl. CPC A61K 8/0254; A61K 8/19; A61K 8/25; A61K 8/26; A61Q 1/06; A61Q 1/08; A61Q 1/10; A61Q 1/12; A61K 2800/43; A61K 2800/621; A61K 2800/651

(57) ABSTRACT

A composition contains (a) at least one flake-type aluminum pigment and (b) at least one flake-type black pigment. One embodiment is a composition for a make-up on a keratinous substance such as skin, and another embodiment is a cosmetic process. The (a) flake-type aluminum pigment may contain an aluminum simple substance.

### TECHNICAL FIELD

The present invention relates to a composition comprising at least one flake-type aluminum pigments and at least one flake-type black pigment, for a make-up for a keratinous substance such as skin, and a cosmetic process using the same.

### BACKGROUND ART

Cosmetic make-up compositions comprising pigments and colorants for keratinous substances are widely used as, for example, a foundation powder, a finishing powder, a face powder, a blusher, and an eyeshadow. These compositions are used to provide keratinous substances with an attractive appearance. Therefore, for cosmetic compositions to serve as make-up cosmetics, aesthetic effects, such as effects to provide natural finish and three-dimensional appearance effects to keratinous substances, preferably skin, in particular facial skin, are very important. In order to impart such aesthetic effects to cosmetic compositions, the use of several effect pigments in the compositions has been proposed.

For example, JP-A-H7-228509 discloses a make-up cosmetic composition containing aluminum-treated powder produced by coating plate-like inorganic powder with aluminum powder by a mechanical method.

JP-A-2010-83727 also discloses a scaly iron oxide particle having a magnetite-structure, an average length of 5-30 μm in a direction of a plate surface, and an average length of 0.05-0.5 μm in a thickness direction.

JP-A-2010-222295 also discloses a cosmetic composition comprising a flake-type black iron oxide having an average particle diameter of 5-100 μm and a thickness of 0.2-0.3 μm, wherein the surface of the flake-type black iron oxide is coated with at least one of hydrous or anhydrous inorganic oxides of silicon, aluminum, titanium, zirconium, tin or the like.

JP-T-2010-513619 also discloses a platelet-like aluminum pigments having a narrow thickness distribution and at least partially coated with lubricant, wherein the pigments have a) a mean thickness h₅₀ of from 15 nm to 75 nm as determined by a scanning electron microscope thickness count, b) a relative breadth of thickness distribution Δh of from 30% to less than 70%, as determined by a scanning electron microscope thickness count and as calculated on the basis of the corresponding cumulative breakthrough curve of the relative frequencies of occurrence, according to the formula Δh=100×(h₉₀−h₁₀)/h₅₀, and c) an X-ray diffractogram, measured on pigments in substantially plane-parallel orientation, having one or two main peaks which do not correspond to the [111] reflexes.

JP-A-2005-307155 also discloses lustrous black interference pigments comprising a mixture of different substrates having (A) a coating of Fe₃O₄, (B) a colorless coating consisting of one or more layers each having a refractive index of n≤1.8, (C) optionally a coating with an absorbent, high-refractive-index material which only covers part of the underlying surface, and optionally (D) an outer protective layer.

JP-T-2014-508211 also discloses a black effect pigment comprising a) at least a partial layer of SnO₂ and/or hydrated SnO₂ on a substrate, b) at least a partial layer of Fe₃O₄, and c) optionally, a further layer of metal oxide, preferably the metal oxide layer is transparent, wherein the at least partial layer of Fe₃O₄ may further contain ferric hydroxide and Fe₂O₃.

However, there is still a demand for cosmetic make-up products with improved cosmetic effects. In particular, there is still a demand for cosmetic make-up products providing aesthetic effects such as effects to provide natural finish and three-dimensional appearance effects to keratinous substances, preferably skin, in particular facial skin around the eyes.

### DISCLOSURE OF INVENTION

An objective of the present invention is to provide a composition for make-up use, which can provide improved aesthetic effects, such as effects to provide natural finish and a three-dimensional appearance effect to keratinous substances, preferably skin, in particular facial skin around the eyes.

The above objective of the present invention can be achieved by a composition comprising:
(a) at least one flake-type aluminum pigment; and
(b) at least one flake-type black pigment.

The (a) flake-type aluminum pigment may comprise an aluminum simple substance.

The (a) flake-type aluminum pigment may have an average particle size of 1 μm or more, preferably 3 μm or more, and more preferably 5 μm or more, and in particular 8 μm or more, and may have an average particle size of 80 μm or less, preferably 60 μm or less, and more preferably 40 μm or less.

The (a) flake-type aluminum pigment may have a thickness of 0.01 μm or more, and preferably 0.03 μm or more, and may have a thickness of 5 μm or less, and preferably 3 μm or less.

The (a) flake-type aluminum pigment may be coated with at least one metal oxide which may be selected from silica, aluminum oxides, titanium oxides, zirconium oxides, cerium oxide, chromium oxide, and iron oxides.

The amount of the (a) flake-type aluminum pigment(s) may be 3% by weight or more, preferably 5% by weight or more, and more preferably 7% by weight or more, and may be 97% by weight or less, preferably 80% by weight or less, more preferably 50% by weight or less, and even more preferably 30% by weight or less, relative to the total weight of pigments and colorants included in the composition.

The (b) flake-type black pigment may be chosen from black iron oxide, graphite, and mica or synthetic coated with back iron oxide or carbon black.

The (b) flake-type black pigment may have an average particle size of 3 μm or more, preferably 5 μm or more, and more preferably 7 μm or more, and may have an average particle size of 70 μm or less, preferably 50 μm or less, and more preferably 30 μm or less. The (b) flake-type black pigment may have a thickness of 0.01 μm or more and 5 μm or less. The amount of the (b) flake-type black pigment(s) may be 3% by weight or more, preferably 5% by weight or more, and more preferably 7% by weight or more, and may be 97% by weight or less, preferably 80% by weight or less, more preferably 50% by weight or less, and even more preferably 30% by weight or less, relative to the total weight of pigments and colorants included in the composition.

The weight ratio of the (a) flake-type aluminum pigment to the (b) flake-type black pigment in the composition may range 1:9 to 9:1, preferably 1:7 to 7:1, and more preferably 1:5 to 5:1, and even more preferably 1:4 to 4:1.

The composition according to the present invention may be for use as a make-up powder, such as a powder foundation, a liquid foundation, a finishing powder or a face powder, a body makeup product, a blusher, a cheek, a lipstick, an eyeshadow primer, a face contouring, and eyeshadow, in particular an eyeshadow.

The present invention also relates to a cosmetic process for make-up for keratinous substances, preferably skin, in particular skin around the eyes, comprising a step of applying onto the keratinous substances the composition according the present invention.

### BEST MODE FOR CARRYING OUT THE INVENTION

After diligent research, the inventors have surprisingly found out that a combination of the (a) at least one flake-type aluminum pigment and the (b) at least one flake-type black pigment can provide an improved three-dimensional appearance and natural finish to keratinous substances, such as facial skin, in particular skin around the eyes, and thus they have completed the present invention.

Thus, the composition according to the present invention comprises:
(a) at least one flake-type aluminum pigment; and
(b) at least one flake-type black pigment.

Hereafter, the composition according to the present invention will be described in a detailed manner.

### [Composition]

The composition according to the present invention comprises (a) at least one flake-type aluminum pigment and (b) at least one flake-type black pigment. Each ingredient is explained in a detailed manner as below.

### (Flake-Type Aluminum Pigment)

The composition according to the present invention comprises (a) at least one flake-type aluminum pigment. Two or more (a) flake-type aluminum pigments may be used in combination. Thus, a single type of flake-type aluminum pigment or a combination of different types of flake-type aluminum pigments may be used.

The term “pigment” here should be understood as meaning white or coloured, mineral or organic particles that are insoluble in a medium in the composition.

The term “flake-type” here can be well understood in the art. The term “flake-type” may be used interchangeably with flake, flake-like, plate-like, platelet, platy, and flaky.

The (a) flake-type aluminum pigment comprises an aluminum simple substance. The term “aluminum simple substance” here can mean a substance which is made up with only aluminum element. The (a) flake-type aluminum pigment may partially or totally comprise an aluminum simple substance. In one embodiment of the present invention, the (a) flake-type aluminum pigment comprises a core of the aluminum simple substance.

The (a) flake-type aluminum pigment may comprise aluminum alloys or aluminum compounds other than the simple aluminum substances. The (a) flake-type aluminum pigment may be a mixture of the aluminum simple substance, the aluminum alloy, and aluminum compounds. The aluminum alloy is not limited to, but for example mention can be made of aluminum alloy contains iron, manganese, copper, vanadium, chromium, nickel, cobalt, silicon, magnesium, zinc, and/or titanium. The aluminum compound is not limited to, but for example mention can be made of oxides and/or hydroxides of aluminum.

The (a) flake-type aluminum pigment may be present in the composition as a different ingredient from the (b) flake-type black pigment. In one embodiment of the present invention, the (a) flake-type aluminum pigment does not bind chemically or physically to the (b) flake-type black pigment.

The (a) flake-type aluminum pigment may have an average particle size of 1 μm or more, preferably 3 μm or more, and more preferably 5 μm or more, and in particular 8 μm or more, and/or may have an average particle size of 80 μm or less, preferably 60 μm or less, and more preferably 40 μm or less. The term “average particle size” used herein represents a number-average size mean diameter which is given by the statistical particle size distribution to half of the population, referred to as D50. For example, the number-average size mean diameter can be measured by a laser diffraction particle size distribution analyzer, such as Mastersizer from Malvern Corp.

The thickness of the (a) flake-type aluminum pigment is not limited, but in general may be 0.01 μm or more, preferably 0.03 μm or more, and more preferably 0.05 μm or more, and/or may be 5 μm or less, preferably 3 μm or less, and more preferably 1 μm or less. Here, the term “thickness” can mean an average thickness. The average thickness of the pigments in the present invention can be measured by a well-known technique in the art, for example, by using an atomic force microscope (AFM).

The (a) flake-type aluminum pigment may or may not be coated. In one preferred embodiment, the (a) flake-type aluminum pigment has at least one coating. The coating may be inorganic substance and/or organic substance.

The inorganic coating may be selected from metal oxides, such as silica, aluminum oxides, titanium oxides, zirconium oxides, cerium oxide, chromium oxide, and iron oxides, and metal hydroxides, such as aluminum hydroxide.

The organic coating may be selected from fatty acids or salts thereof (such as sodium, potassium, zinc, iron or aluminum salts), fatty alcohols, lecithin, amino acids, polysaccharides, proteins, alkanolamines, waxes such as beeswax, (meth)acrylic polymers, and (per)fluoro compounds.

The coating also may include a hydrophobic agent, such as silanes, preferably a C₁-C₂₀-alkylsilane, more preferentially a tri(C₁-C₄)alkoxy(C₁-C₁₂)alkyl silane such as triethoxycaprylylsilane, organopolysiloxanes, such as organosilicone, di-organosilicone, dimethicones, hydrogen dimethicone, methicones; polyurethanes, silicone-polyurethanes, and fluoro- or perfluoro-derivatives thereof, fatty acid soaps, C9-15 fluoroalcohol phosphates, acrylate/dimethicone copolymers, mixed C9-C15 fluoroalcohol phosphate/silicone copolymers. Other hydrophobic agents may include isopropyl titanium triisostearate (ITT), ITT and dimethicone (ITT/dimethicone) cross-polymers, ITT and amino acid, ITT/triethoxycaprylylsilane crosspolymer, fatty acids (e.g., stearates), HDI/trimethylol hexyllactone crosspolymer, PEG-8 methyl ether triethoxysilane, aloe, jojoba ester, and Magnesium Myristate (MM).

In one preferred embodiment of the present invention, the (a) flake-type aluminum pigment is coated with at least one metal oxide, such as silica, aluminum oxides, titanium oxides, zirconium oxides, and iron oxides, and preferably silica. In one embodiment of the present invention, the (a) flake-type aluminum pigment is not coated with organic substances. In another embodiment of the present invention, the (a) flake-type aluminum pigment is not coated with polymers.

As commercially available products of the (a) flake-type aluminum pigment, mention can be made of:
silica-coated aluminum flake sold under the name of Velvet Silver® by TOYAL;
silica-coated aluminum flake sold under the name of Visionaire Bright Silver Sea® by Eckart; and
silica-coated aluminum flake sold under the name of Visionaire Splendid Silver Sea® by Eckart.

The amount of the (a) flake-type aluminum pigment(s) may be 3% by weight or more, preferably 5% by weight or more, and more preferably 7% by weight or more, and/or 97% by weight or less, preferably 80% by weight or less, more preferably 50% by weight or less, and even more preferably 30% by weight or less, relative to the total weight of pigments and colorants included in the composition according to the present invention.

In one embodiment of the present invention, the (a) flake-type aluminum pigment(s) may be 3% by weight or more, preferably 5% by weight or more, and more preferably 7% by weight or more, and/or 97% by weight or less, preferably 80% by weight or less, more preferably 50% by weight or less, and even more preferably 30% by weight or less, relative to the total weight of powder ingredients including fillers included in the composition according to the present invention.

In another embodiment of the present invention, the (a) flake-type aluminum pigment(s) may be 3% by weight or more, and preferably 5% by weight or more, and/or 90% by weight or less, preferably 60% by weight or less, more preferably 30% by weight or less, and even more preferably 15% by weight or less, relative to the total weight of the composition according to the present invention.

In one specific embodiment, when the composition according to the present invention is in a form of a lipstick, for example, the (a) flake-type aluminum pigment(s) can be formulated in an amount of 0.5% by weight to 20% by weight relative to the total weight of the ingredients. In another specific embodiment, when the composition according to the present invention is in a form of a liquid foundation composition, for example, the (a) flake-type aluminum pigment(s) can be formulated in an amount of 0.5% by weight to 20% by weight relative to the total weight of the ingredients. Also, when the composition according to the present invention is in a form of a powder foundation composition, for example, the (a) flake-type aluminum pigment(s) can be formulated in an amount of 2% by weight to 100% by weight relative to the total weight of the ingredients.

### (Flake-Type Black Pigment)

The composition according to the present invention comprises (b) at least one flake-type black pigment. Two or more (b) flake-type black pigments may be used in combination. Thus, a single type of flake-type black pigment or a combination of different types of flake-type black pigments may be used.

The term “black” here may be used to mean substantially black. The term “substantially black” may be used to mean for purposes of this disclosure that the black color is distinguished by sufficient blackness, that is of low L* value, and the “a*” and “b*” values are around the zero point. The black may contain overtones of other colors such as gold, red, green etc. Defined adjustments of the “a*” and “b*” values enables black interference pigments having a gold, green red or blue tint to be obtained.

The (b) flake-type black pigment may be present in the composition as a different ingredient from the (a) flake-type aluminum pigment. In one embodiment of the present invention, the (b) flake-type black pigment does not bind chemically or physically to the (a) flake-type aluminum pigment.

The (b) flake-type black pigment is not particularly limited as long as they show a black color, but may be chosen from a metal oxide, such as black iron oxide, graphite, and carbon black.

The (b) flake-type black pigment can also be selected from flake-like inorganic fillers coated with black pigments such as black iron oxide, graphite, and carbon black. In this embodiment, the flake-like inorganic filler is not particularly limited as long as it has a flake-like shape, but mention can be made of, for example, talc, mica, magnesium aluminum silicate, trimethyl siloxysilicate, kaolin, bentone, calcium carbonate, magnesium hydrogen carbonate, hydroxyapatite, boron nitride, fluorphlogopite, sericite, calcinated talc, calcinated mica, calcinated sericite, synthetic mica, lauroyl lysine, metal soap, bismuth oxychloride, barium sulfate, magnesium carbonate, and mixtures thereof.

Preferentially, the (b) flake-type black pigment is chosen from black iron oxide, in particular Fe₃O₄, graphite, and mica or synthetic coated with back iron oxide or carbon black.

The (b) flake-type black pigment may or may not be coated. The coating may be inorganic substance and/or organic substance.

The inorganic coating may be selected from metal oxides, such as silica, aluminum oxides, titanium oxides, zirconium oxides, cerium oxide, chromium oxide, and iron oxides, and metal hydroxides, such as aluminum hydroxide.

The organic coating may be selected from fatty acids or salts thereof (such as sodium, potassium, zinc, iron or aluminum salts), fatty alcohols, lecithin, amino acids, polysaccharides, proteins, alkanolamines, waxes such as beeswax, (meth)acrylic polymers, and (per)fluoro compounds.

The coating also may include a hydrophobic agent, such as silanes, preferably a C₁-C₂₀-alkylsilane, more preferentially a tri(C₁-C₄)alkoxy(C₁-C₁₂)alkyl silane such as triethoxycaprylylsilane, organopolysiloxanes, such as organosilicone, di-organosilicone, dimethicones, hydrogen dimethicone, methicones; polyurethanes, silicone-polyurethanes, and fluoro- or perfluoro-derivatives thereof, fatty acid soaps, C9-15 fluoroalcohol phosphates, acrylate/dimethicone copolymers, mixed C9-C15 fluoroalcohol phosphate/silicone copolymers. Other hydrophobic agents may include isopropyl titanium triisostearate (ITT), ITT and dimethicone (ITT/dimethicone) cross-polymers, ITT and amino acid, ITT/triethoxycaprylylsilane crosspolymer, fatty acids (e.g., stearates), HDI/trimethylol hexyllactone crosspolymer, PEG-8 methyl ether triethoxysilane, aloe, jojoba ester, and Magnesium Myristate (MM).

The (b) flake-type black pigment may have an average particle size of 3 μm or more, preferably 5 μm or more, and more preferably 7 μm or more, and/or the (b) flake-type black pigment may have an average particle size of 70 μm or less, preferably 50 μm or less, and more preferably 30 μm or less.

The thickness of the (b) flake-type black pigment is not limited, but in general may be 0.01 μm or more, preferably 0.03 μm or more, and more preferably 0.05 μm or more, and/or may be 5 μm or less, preferably 3 μm or less, and more preferably 1 μm or less.

As commercially available products of the (b) flake-type black pigment, mention can be made of:
black flake-type iron oxide sold under the name of BM-200P® by Titan Kogyo;
mica flake coated with black iron oxide sold under the name of COLORONA MICA BLACK® by Merck;
mica flake coated with black iron oxide sold under the name of TIMICA TERRA BLACK® by BASF;
Synthetic mica flake coated with black iron oxide sold under the name of NK-Black® by Nihon Koken Kogyo.

The amount of the (b) flake-type black pigment(s) may be 3% by weight or more, preferably 5% by weight or more, and preferably 7% by weight or more, and/or 97% by weight or less, preferably 80% by weight or less, more preferably 50% by weight or less, and even more preferably 30% by weight or less, relative to the total weight of pigments and colorants included in the composition according to the present invention.

In one embodiment of the present invention, the amount of the (b) flake-type black pigment(s) may be 3% by weight or more, preferably 5% by weight or more, and more preferably 7% by weight or more, and/or 97% by weight or less, preferably 80% by weight or less, more preferably 50% by weight or less, and even more preferably 30% by weight or less, relative to the total weight of powder ingredients including fillers included in the composition according to the present invention.

In another embodiment of the present invention, the amount of the (b) flake-type black pigment(s) may be 3% by weight or more, and preferably 5% by weight or more, and/or 90% by weight or less, preferably 60% by weight or less, more preferably 30% by weight or less, and even more preferably 15% by weight or less, relative to the total weight of the composition according to the present invention.

In another embodiment of the present invention, the weight ratio of the (a) flake-type aluminum pigment to the (b) flake-type black pigment in the composition can be 1:9 to 9:1, preferably 1:7 to 7:1, and more preferably 1:5 to 5:1, and even more preferably 1:4 to 4:1.

### (Other Ingredients)

### Colorant

The compositions according to the invention may comprise at least one colorant, which is different from the (a) flake-type aluminum pigment and the (b) flake-type black pigment. Two or more colorants may be used in combination. Thus, a single type of colorant or a combination of different types of colorants may be used.

The term “colorant” should be understood as encompassing pigments, nacres, and reflective particles, and mixtures thereof. The colorant may be represented as dyestuff.

### Pigments

The pigments may be white or colored, and mineral and/or organic.

Among the mineral pigments that may be mentioned are titanium dioxide, such as pigmentary titanium dioxide rutile type, optionally surface-treated, zirconium oxide or cerium oxide, and also zinc oxide, iron (black, yellow or red) oxide or chromium oxide, manganese violet, ultramarine blue, chromium hydrate and ferric blue, and metal powders, for instance aluminum powder and copper powder.

The organic pigments may be chosen from the materials below, and mixtures thereof:
cochineal carmine,
organic pigments of azo dyes, anthraquinone dyes, indigoid dyes, xanthene dyes, pyrene dyes, quinoline dyes, triphenylmethane dyes and fluorane dyes.

Among the organic pigments, mention may be made especially of the D&C certified pigments known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, and FD&C Yellow No. 6.

The chemical materials corresponding to each of the organic dyestuffs mentioned previously are mentioned in the publication “International Cosmetic Ingredient Dictionary and Handbook”, 1997 edition, pages 371 to 386 and 524 to 528, published by The Cosmetic, Toiletries and Fragrance Association, the content of which is incorporated into the present patent application by reference.

### Nacres

The term “nacres” should be understood as meaning colored particles of any form, which may or may not be iridescent, especially produced by certain molluscs in their shell, or alternatively synthesized, and which have a color effect via optical interference.

Examples of nacres that may be mentioned include nacreous pigments such as titanium mica coated with an iron oxide, mica coated with bismuth oxychloride, titanium mica coated with chromium oxide, and nacreous pigments based on bismuth oxychloride. They may also be mica particles at the surface of which are superposed at least two successive layers of metal oxides and/or of organic dyestuffs.

As illustrations of nacres that may be introduced into the composition, mention may be made of the gold-colored nacres sold especially by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company BASF under the name Super bronze (Cloisonne); the orange nacres sold especially by the company BASF under the name Orange 363C (Cloisonne) and Orange MCR 101 (Cosmica) and by the company Merck under the name Passion orange (Colorona) and Matte orange (17449) (Microna); the brown nacres sold especially by the company BASF under the name Nu-antique copper 340XB (Cloisonne) and Brown CL4509 (Chromalite); the nacres with a copper tint sold especially by the company BASF under the name Copper 340A (Timica); the nacres with a red tint sold especially by the company Merck under the name Sienna fine (17386) (Colorona); the nacres with a yellow tint sold especially by the company BASF under the name Yellow (4502) (Chromalite); the red nacres with a gold tint sold especially by the company BASF under the name Sunstone G012 (Gemtone); the pink nacres sold especially by the company BASF under the name Tan opale G005 (Gemtone); the black nacres with a gold tint sold especially by the company BASF under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

The nacres used in the present invention are also listed in the following table. These nacres shows a reflection color of blue, green or a color travel.

| Reflection | SUPPLIER | COMMERCIAL | |
|---|---|---|---|
| color | NAME | RANGE NAME | COMMERCIAL FULL NAME (July 2017) |
| Blue | BASF | FLAMENCO | FLAMENCO BLUE 620C |
| Blue | BASF | CLOISONNE | CLOISONNE BLUE 626C |
| Blue | MERCK | COLORONA | COLORONA DARK BLUE |
| Blue | SUN | SUNSHINE | SUNSHINE STAR BLUE C83-3405 |
| Blue | SUN | INTENZA | C91-4251 INTENZA BLUE LAGOON |
| Color travel | MERCK | XIRONA | XIRONA CARIBBEAN BLUE |
| Color travel | BASF | REFLECKS | REFLECKS MULTIDIMENSIONS |
| | | MULTIDIMENSIONS | SHIFTING SAPPHIRE G680D |
| Color travel | BASF | REFLECKS | REFLECKS MULTIDIMENSIONS |
| | | MULTIDIMENSIONS | TRANSFORMING TEAL G780D |
| Color travel | SUN | SUNSHINE | SUNSHINE NOTHERN LIGHTS C92-4260 |
| Color travel | SUN | SUNSHINE | SUNSHINE DAYBREAK BLUE C92-4250 |
| Green | BASF | DUOCROME | DUOCROME BG 826C |
| Green | BASF | CLOISONNE | CLOISONNE SUPER GREEN 827C |
| Green | BASF | GEMTONE | GEMTONE EMERALD G 002 |
| Green | ECKART | SYNCRYSTAL | SYNCRYSTAL JADE |

As further examples of nacres, mention may also be made of particles comprising a borosilicate substrate coated with titanium oxide.

Particles having a glass substrate coated with titanium oxide are especially sold under the name Metashine MT1080RY by the company NSG (Nippon Sheet Glass).

Finally, examples of nacres that may also be mentioned include polyethylene terephthalate flakes, especially those sold by the company Meadowbrook Inventions under the name Silver 1P 0.004X0.004 (silver flakes).

### Reflective Particles

The term “reflective particles” denotes particles whose size, structure, especially the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, points of overbrightness that are visible to the naked eye, i.e. more luminous points that contrast with their environment by appearing to sparkle.

These particles may have varied forms and may especially be in platelet or globular form, in particular in spherical form.

The reflective particles, whatever their form, may or may not have a multilayer structure and, in the case of a multilayer structure, may have, for example, at least one layer of uniform thickness, in particular of a reflective material.

When the reflective particles do not have a multilayer structure, they may be composed, for example, of metal oxides, especially titanium or iron oxides obtained synthetically.

When the reflective particles have a multilayer structure, they may comprise, for example, a natural or synthetic substrate, especially a synthetic substrate at least partially coated with at least one layer of a reflective material, especially of at least one metal or metallic material. The substrate may be made of one or more organic and/or inorganic materials.

More particularly, it may be chosen from glasses, ceramics, graphite, metal oxides, aluminas, silicas, silicates, especially aluminosilicates and borosilicates, and synthetic mica, and mixtures thereof, this list not being limiting.

The reflective material may comprise a layer of metal or of a metallic material.

Again as an example of reflective particles comprising a mineral substrate coated with a layer of metal, mention may also be made of particles comprising a silver-coated borosilicate substrate.

Particles with a silver-coated glass substrate, in the form of platelets, are sold under the name Microglass Metashine REFSX 2025 PS by the company NSG. Particles with a glass substrate coated with nickel/chromium/molybdenum alloy are sold under the name Crystal Star GF 550 and GF 2525 by the company TOYAL.

Use may also be made of particles comprising a metallic substrate such as silver, iron, chromium, nickel, molybdenum, gold, copper, zinc, tin, manganese, steel, bronze or titanium, said substrate being coated with at least one layer of at least one metal oxide such as titanium oxide, aluminum oxide, iron oxide, cerium oxide, chromium oxide or silicon oxides, and mixtures thereof.

The amount of the colorant(s) may be 30% by weight or more, preferably 50% by weight or more, and more preferably 65% by weight or more, and/or 95% by weight or less, preferably 85% by weight or less, and more preferably 80% by weight or less, relative to the total weight of pigments and colorants included in the composition according to the present invention.

In one embodiment of the present invention, the colorant(s) may be 30% by weight or more, and preferably 50% by weight or more, and more preferably 65% by weight or more, and/or 95% by weight or less, preferably 85% by weight or less, and more preferably 80% by weight or less, relative to the total weight of powder ingredients including fillers included in the composition according to the present invention.

In another embodiment of the present invention, the amount of the colorant(s) may be 20% by weight or more, preferably 30% by weight or more, and more preferably 40% by weight or more, and/or 90% by weight or less, preferably 80% by weight or less, more preferably 75% by weight or less, and even more preferably 70% by weight or less, relative to the total weight of the composition according to the present invention.

### Filler

The composition according to the present invention may preferably include at least one filler. Preferably, the composition according to the present invention may include one or more organic and/or inorganic fillers, in addition to the (a) flake-type aluminum pigment and (b) flake-type black pigment. If two or more fillers are used, they may be the same or different.

The term “fillers” should be understood as meaning mineral or synthetic particles of any shape which are insoluble in the medium of the composition.

The fillers may be of any shape: platelet-shaped, spherical or oblong, irrespective of the crystallographic form (for example lamellar, cubic, hexagonal, orthorhombic, etc.).

As the inorganic fillers, mention may be made of talc, mica, silica, magnesium aluminum silicate, trimethyl siloxysilicate, kaolin, bentone, calcium carbonate, magnesium hydrogen carbonate, hydroxyapatite, boron nitride, fluorphlogopite, synthetic fluorphlogopite, sericite, calcinated talc, calcinated mica, calcinated sericite, synthetic mica, lauroyl lysine, metal soap, bismuth oxychloride, barium sulfate, magnesium carbonate, and mixtures thereof, optionally hydrophilic- or hydrophobic-treated.

As the organic fillers, mention may be made of (meth)acrylic or (meth)acrylate powders, for example, polymethylmethacrylate powders; polyacrylonitrile powders; organopolysiloxane powders, polyamide powders, poly-ß-alanine powders and polyethylene powders, polytetrafluoroethylene powders, lauroyllysine, rice or corn starch, such as corn starch: INCI name: Zea Mays (Corn) Starch, tetrafluoroethylene polymer powders, hollow polymer microspheres, for example comprising an (alkyl)acrylate, metal soaps derived from organic carboxylic acids containing from 8 to 22 carbon atoms, for example zinc stearate, magnesium stearate, lithium stearate, zinc laurate, magnesium myristate, silicone powder, such as silicone resin powder, polyorganosilsesquioxane powder, vinyl dimethicone/methicone silsesquioxane crosspolymer powder, and a polymethylsilsesquioxane powder, polyurethane powders, carnauba microwaxes, synthetic microwaxes, polyamide powder, such as those listed under the CTFA name of “Nylon 12” or “Nylon 6”, and mixtures thereof.

The filler may be present in a content ranging from 1% to 30% by weight, preferably from 2% to 20% by weight and more preferably from 3% to 10% by weight, relative to the total weight of the composition.

### Oil

The composition according to the present invention may preferably include at least one oil. The oil can be volatile or non-volatile. Preferably, the oil is a non-volatile oil. If two or more oils are used, they may be the same or different.

The non-volatile hydrocarbon-based oil may be selected from:
hydrocarbon-based oils of animal origin, such as perhydrosqualene,
hydrocarbon-based oils of plant origin, such as phytostearyl esters, such as phytostearyl oleate, phytostearyl isostearate and lauroyl/octyldodecyl/phytostearyl glutamate, triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, winter squash oil, millet oil, barley oil, quinoa oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camelina oil, canola oil, carrot oil, safflower oil, flax oil, rapeseed oil, cottonseed oil, coconut oil, marrow seed oil, wheatgerm oil, jojoba oil, lily oil, macadamia oil, corn oil, meadowfoam oil, monoi oil, hazelnut oil, apricot kernel oil, walnut oil, olive oil, evening primrose oil, palm oil, blackcurrant seed oil, kiwi seed oil, grapeseed oil, pistachio oil, winter squash oil, pumpkin oil, quinoa oil, musk rose oil, sesame oil, soybean oil, sunflower oil, castor oil and watermelon oil, and mixtures thereof, or alternatively caprylic/capric acid triglycerides,
linear or branched hydrocarbons of mineral or synthetic origin, such as liquid paraffins and derivatives thereof, petroleum jelly, mineral oil, polydecenes, polybutenes, hydrogenated polyisobutene such as Parleam, and squalane;
synthetic ethers containing from 10 to 40 carbon atoms;
synthetic esters, for instance oils of formula R₁COOR₂, in which R₁ represents a linear or branched fatty acid residue containing from 1 to 40 carbon atoms, and R₂ represents a hydrocarbon-based chain that is especially branched, containing from 1 to 40 carbon atoms provided that R₁+R₂≥10. The esters may be chosen especially from fatty acid esters of alcohols, for example, cetyl esters, in particular, cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoates, in particular C₁₂-C₁₅ alkyl benzoates, polyethylene glycol diheptanoate, propylene glycol 2-diethylhexanoate, and mixtures thereof, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate
fluoro oils that are optionally partially hydrocarbon-based and/or silicone-based, for instance fluorosilicone oils, fluoropolyethers and fluorosilicones;
fatty alcohols that are saturated or unsaturated, linear or branched, and contain 6 to 30 carbon atoms and more particularly from 8 to 30 carbon atoms: mention may be made of cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
silicone oils, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane; and phenyl silicone oils such as phenyl trimethicone, and
mixtures thereof.

The oil may be present in a content ranging from 2% to 40% by weight, preferably from 5% to 35% by weight, and more preferably from 10% to 30% by weight relative to the total weight of the composition.

### Binder Polymer

The composition according to the present invention may preferably include at least one binder polymer. If two or more binder polymers are used, they may be the same or different.

The binder polymer, which can be used in the present invention, may be homopolymer or copolymer having a monomer unit selected from (meth)acrylate, styrene, acrylamide, acrylonitrile, vinyl acetate, olefins, tetrafluoroethylene and divinylbenzene, more preferably binder polymer selected from polystyrene, polyethylene, poly-2-alanine, polytetrafluoroethylene, poly(vinyl acetate), poly(methyl styrene), poly(acrylamide), poly(acrylonitrile), poly(vinyl chloride), polyacrylate, polymethacrylate, polyurethane, silicone resin, nylon, polyorganosiloxane, polysaccharides such as cellulose, copolymer of styrene and C₁-C₁₆ alkyl(meth)acrylate, copolymer of styrene and acrylamide, copolymer of styrene and acrylonitrile, copolymer of styrene and vinyl acetate, copolymer of acrylamide and C₁-C₁₆ alkyl (meth)acrylate, copolymer of acrylonitrile and C₁-C₁₆ alkyl (meth)acrylate, copolymer of acrylonitrile and acrylamide, terpolymer of styrene, acrylonitrile and acrylamide, copolymer of styrene/butadiene, styrene/acrylic acid, styrene/vinylpyrrolidone vinylidene chloride/acrylonitrile, butadiene/acrylonitrile, and combinations thereof.

It is preferable that the binder polymer be oil soluble or oil dispersible.

The binder polymer is also preferably film-forming, i.e. it is capable of forming a film when applied to the skin and the like.

The binder polymer may especially be a diblock, triblock, multiblock, radial or star copolymer, or mixtures thereof, and preferably a diblock copolymer.

Examples of olefins that may be mentioned include ethylenic carbide monomers, especially containing one or two ethylenic unsaturations and containing from 2 to 5 carbon atoms, such as ethylene, propylene, butadiene, isoprene or pentadiene.

According to one preferred embodiment, the binder polymer is hydrogenated to reduce the residual ethylenic unsaturations after the polymerization of the monomers.

In particular, the binder polymer is a copolymer, optionally hydrogenated, containing styrene blocks and ethylene and/or C3-C4 alkylene blocks.

According to one embodiment of the present invention, the binder polymer may be a linear styrene-ethylene/butylene-styrene triblock copolymer or a hydrogenated styrene/butadiene copolymer. Such as triblock copolymer is especially sold under the name Kraton® G1657M by the company Kraton Polymers.

The binder polymer may be present in a content ranging from 0.5% to 20% by weight, preferably from 1% to 10% by weight and more preferably from 1.5% to 5% by weight, relative to the total weight of the composition.

### Additives

The composition according to the present invention may comprise additives as long as they do not impair the effects of the present invention and they are acceptable in cosmetic use. The additives may be selected from anionic, cationic, nonionic or amphoteric polymers; natural or synthetic thickeners; lubricants or dispersants; gelling agents; natural extracts derived from animals or vegetables; waxes; cosmetically acceptable hydrophobic organic solvents; preservatives, such as caprylyl glycol; co-preservatives; bactericides; vitamins or provitamins; fragrances; stabilizers, and mixtures thereof.

The amount of the additives included in the composition according to the present invention is not limited, but may be from 0.01 to 30% by weight relative to the total weight of the composition according to the present invention.

The amount of powder ingredients including pigments, colorants, and fillers in the composition depends on a format of the composition according to the present invention. The amount of powder ingredient can be 10% by weight or more, 30% by weight or more, 50% by weight or more, or 70% by weight or more, and can be 99% by weight or less, 95% by weight or less, 90% by weight or less, 85% by weight or less, or 80% by weight or less.

The composition of the present invention can be a cosmetic make-up composition for a keratinous substance, such as skin, for example, of the face, neck and body, in particular skin around the eyes, and more preferably a skin cosmetic make-up composition. The composition according to the present invention can be provided in the form of a compact or pressed powder, blusher or a loose powder. The composition of the present invention can be also provided in a form of lipstick, a liquid foundation, and a powder foundation.

The composition according to the present invention can provide an improved natural and three-dimensional appearance to keratinous substances, in particular skin around the eyes, while it exhibits an improved application property, i.e. spreadability. Therefore, the composition according to the present invention can provide a natural appearance and more defined facial features.

It should be noted that the use of the composition according to the present invention can create a three-dimensional appearance on the face, in particular skin around the eyes, and thus it is not required to use different types of powder cosmetics which has been conventionally required to create the three-dimensional appearance. In addition, the use of the present invention does not require a high level of skill for consumers to create the three-dimensional appearance on their faces, in particular skin around the eyes. The present invention can provide a three-dimensional appearance and natural finish on the face, in particular skin around the eye in a simple and easy way.

Accordingly, the composition according to the present invention is very suitable for use as a make-up cosmetics, such as a powder foundation, a liquid foundation, a finishing powder or a face powder, a body makeup product, a blusher, a cheek, a lipstick, an eyeshadow primer, a face contouring, and eyeshadow, in particular an eyeshadow.

In one embodiment of the present invention, the composition according to the present invention is preferably anhydrous or contains less than 3% by weight of water or hydrophilic solvent(s) and preferably less than 1% by weight of water or hydrophilic solvent(s), relative to the total weight of the composition. The term “anhydrous” especially means that water is preferably not deliberately added to the composition, and more preferably is not present in the composition, but may be present in trace amount in the various ingredients used in the composition. The term “hydrophilic” here means substances having a solubility of at least 1 g/L, preferably at least 10 g/L, and more preferably at least 100 g/L, in water at room temperature (25° C.) and atmosphere pressure (105 Pa).

### [Cosmetic Process]

The present invention also relates to a cosmetic process including a step of applying to keratinous substances, such as skin, preferably facial skin, in particular skin around the eyes, the composition according to the present invention. The composition can be picked up with an applicator, such as a sponge, puff, or brush, by rubbing off the powder. The composition can also be simply picked up with a finger by rubbing off the powder. The composition can then be moved from the applicator to the skin by contacting the applicator on the skin.

The composition used according to the present invention is preferably intended to be used as a leave-on type cosmetic composition. The term “leave-on” means a composition that is not intended to be washed off or removed immediately after application.

Since the composition according to the present invention can provide an improved natural and three-dimensional appearance to keratinous substances, such as the face, in particular skin around the eyes, while it shows an improved application property, i.e. spreadability, the cosmetic process according to the present invention can provide keratinous substances, in particular skin around the eyes, with an attractive appearance of a natural look and more defined features.

The present invention also relates to a use of a combination of the (a) flake-type aluminum pigment and the (b) flake-type black pigment as a make-up active in a cosmetic composition, such as a powder foundation, a liquid foundation, a finishing powder or a face powder, a body makeup product, a blusher, a cheek, a lipstick, an eyeshadow primer, a face contouring, and eyeshadow, in particular an eyeshadow.

### Examples

The present invention will be described in a more detailed manner by way of examples. However, these examples should not be construed as limiting the scope of the present invention.

### [Composition]

Eyeshadow compositions according to Examples and Comparative Examples were prepared by mixing the ingredients listed in Table 1, and then pressing with a press machine to obtain pressed powder format. The aluminum flake used in the examples was silica-coated aluminum flake sold under the name of Velvet Silver® obtained from TOYAL. This pigment has 13 μm of an average particle size. The Flake-type black iron oxide used in the examples was black flake-type iron oxide sold under the name of BM-200P® obtained from Titan Kogyo. This pigment has 10 to 20 μm of an average particle size. The mica coated with black iron oxide was mica flake coated with black iron oxide sold under the name of COLORONA MICA BLACK® obtained from Merck. This pigment has 10 to 60 μm of an average particle size.

The compositions are shown in Table 1. In Table 1, all components are based on “% by weight” as active raw materials.

### [Evaluation]

The compositions according to Examples 1 to 3 and Comparative Examples 1 to 4 were evaluated with respect to three key properties of a spreadability, a three-dimensional appearance effect, and natural finish by 10 monitors. Each of the compositions according to Examples 1 to 3 and Comparative Examples 1 to 4 was applied on the monitors' eye lid with a brush by the monitors themselves. The monitors evaluated if they were able to perceive a good spreadability, a three-dimensional appearance effect, and natural finish after application. Table 1 shows how many people perceived each effect.

TABLE 1

| | | | | Comp. | Comp. | Comp. | Comp. |
|---|---|---|---|---|---|---|---|
| Ingredients | Ex. 1 | Ex. 2 | Ex. 3 | Ex. 1 | Ex. 2 | Ex. 3 | Ex. 4 |
| Nylon-12 | 2.9 | 2.9 | 2.9 | 2.9 | 2.9 | 2.9 | 2.9 |
| Boron Nitride | 1.8 | 1.8 | 1.8 | 1.8 | 1.8 | 1.8 | 1.8 |
| Synthetic | 0.25 | 0.25 | 0.25 | 0.25 | 0.25 | 0.25 | 0.25 |
| Fluorphlogopite | | | | | | | |
| TiO₂-coated mica | 50 | 50 | 60 | 50 | 50 | 60 | 60 |
| pigment | | | | | | | |
| Aluminum flake | 10 | 10 | 5 | 10 | — | — | 10 |
| Flake-type black iron | 10 | — | 5 | — | 10 | 10 | — |
| oxide | | | | | | | |
| Mica coated with black | — | 10 | — | — | — | — | — |
| iron oxide | | | | | | | |
| Black iron oxide | — | — | — | 10 | — | — | — |
| TiO₂-coated mica | — | — | — | — | 10 | — | — |
| Hydrogenated | 2.45 | 2.45 | 2.45 | 2.45 | 2.45 | 2.45 | 2.45 |
| Styrene/Butadiene | | | | | | | |
| Copolymer | | | | | | | |
| C12-15 Alkyl Benzoate | 14.7 | 14.7 | 14.7 | 14.7 | 14.7 | 14.7 | 14.7 |
| Phenyl Trimethicone | 7.4 | 7.4 | 7.4 | 7.4 | 7.4 | 7.4 | 7.4 |
| Caprylyl Glycol | 0.5 | 0.5 | 0.5 | 0.5 | 0.5 | 0.5 | 0.5 |
| Evaluation | | | | | | | |
| Spreadability | +++ | +++ | +++ | +++ | ++ | ++ | ++ |
| Three-Dimensional | +++ | +++ | ++ | ++ | ++ | + | + |
| Appearance | | | | | | | |
| Natural Finish | +++ | ++ | +++ | + | ++ | + | + |

+++: >7 persons perceived the effect
++: 4-5 persons perceived the effect
+: <3 persons perceived the effect

As can be seen from the evaluation results in Table 1, the composition according to Examples 1 to 3 including a combination of the flake-type aluminum pigment and the flake-type black pigment exhibited a superior three-dimensional appearance effect, natural finish, and an improved spreadability.

On the other hand, the compositions according to Comparative Examples 1 to 4, which do not comprise the combination of the flake-type aluminum pigment and the flake-type black pigment, could not provide a good three-dimensional appearance effect, natural finish, nor an improved spreadability.

Therefore, it can be concluded that the composition according to the present invention is very preferable for use as a make-up powder, such as a powder foundation, a liquid foundation, a finishing powder or a face powder, a body makeup product, a blusher, a cheek, a lipstick, an eyeshadow primer, a face contouring, and eyeshadow, in particular an eyeshadow.

1. A composition comprising:
(a) at least one flake-type aluminum pigment; and
(b) at least one flake-type black pigment.

2. The composition according to claim 1, wherein the (a) flake-type aluminum pigment comprises an aluminum simple substance.

3. The composition according to claim 1, wherein the (a) flake-type aluminum pigment has an average particle size of 1 μm or more and has an average particle size of 80 μm or less.

4. The composition according to claim 1, wherein the (a) flake-type aluminum pigment has a thickness of 0.01 μm or more and has a thickness of 5 μm or less.

5. The composition according to claim 1, wherein the (a) flake-type aluminum pigment is coated with at least one metal oxide selected from the group consisting of silica, aluminum oxides, titanium oxides, zirconium oxides, cerium oxide, chromium oxide, and iron oxides.

6. The composition according to claim 1, wherein the amount of the (a) flake-type aluminum pigment (s) is 3% by weight or more and is 97% by weight or less, relative to the total weight of pigments and colorants included in the composition.

7. The composition according to claim 1, wherein the (b) flake-type black pigment is chosen from black iron oxide, graphite, and mica or synthetic coated with back iron oxide or carbon black.

8. The composition according to claim 1, wherein the (b) flake-type black pigment has an average particle size of 3 μm or more and has an average particle size of 70 μm or less.

9. The composition according to claim 1, wherein the (b) flake-type black pigment has a thickness of 0.01 μm or more and 5 μm or less.

10. The composition according to claim 1, wherein the amount of the (b) flake-type black pigment (s) is 3% by weight or more and is 97% by weight or less, relative to the total weight of pigments and colorants included in the composition.

11. The composition according to claim 1, wherein the weight ratio of the (a) flake-type aluminum pigment to the (b) flake-type black pigment in the composition ranges 1:9 to 9:1.

12. The composition according to claim 1, wherein the composition is a make-up powder, a liquid foundation, a finishing powder or a face powder, a body makeup product, a blusher, a cheek, a lipstick, an eyeshadow primer, a face contouring, or eyeshadow.

13. A cosmetic process for make-up for keratinous substances, comprising applying onto the keratinous substances the composition according to claim 1.
